(19) United States
(12) Patent Application Publication
(10) Pub. No.: US 2016/0244404 A1
(43) Pub. Date: Aug. 25, 2016

(54) OPTICAL ISOMERS OF (+) AND (-)-BENZHYDRYL UREAS AND (+) AND (-)-1-[(3-CHLOROPHENYL)-PHENYL-METHYL] UREA, A PHARMACEUTICAL COMPOSITION BASED THEREON AND A METHOD FOR PRODUCING SAID OPTICAL ISOMERS

(71) Applicant: OBSCHESTVO S OGRANICHENNOY OTVETSTVENNOSTIYU "SINTEGAL", g. Tomsk (RU)

(72) Inventors: Vladimir Nikolaevich HUDOLEY, p. Timiryazevo (RU); Aleksandr Sergeevich DOROSHENKO, pos. Zonalnaya stantsiya (RU); Vikto Dmitrievich FILIMONOV, Tomsk (RU); Viktoria Viktorovna SHTRYKOVA, Seversk (RU); Vera Yurievna KUKSIONOK, Tomsk (RU)

(21) Appl. No.: 14/905,961

(22) PCT Filed: Mar. 5, 2014
(86) PCT No.: PCT/RU2014/000147

§ 371 Date: Jan. 18, 2016

(30) Foreign Application Priority Data
Jul. 18, 2013 (RU) 2013133610

(51) Int. Cl. C07C 275/24; C07C 273/18

(52) U.S. Cl. CPC C07C 275/24; C07C 273/1809

(57) ABSTRACT

The invention relates to novel substances, and more particularly to optical isomers of (+) and (−)-benzhydryl ureas of formula (I) and (+) and (−)-1-[(3-chlorophenyl)-phenyl-methyl]urea, a pharmaceutical composition based thereon, and a method for producing said optical isomers and for using same on the basis of the different therapeutic activity exhibited. (I) Where R≠R′ and are selected from the group comprising hydrogen, alkyl, halogen, nitro, amino, alkylamino and hydroxy groups and are situated in the ortho-, para- or meta-positions of the benzene rings. When racemic Halodif 1-[(3-chlorophenyl)-phenyl-methyl]urea was separated using the method according to the invention, optical isomers of (+) and (−)-1-[(3-chlorophenyl)-phenyl-methyl]urea (Halodif isomers) with different degrees of therapeutic activity were produced.

### FIELD OF THE INVENTION

The present invention relates to new substances, i.e., to optical isomers of (+) and (−)-benzhydrol ureas represented by the general formula (I), halodif ((+) and (˜)-1-[(3-chlorophenyl)-phenyl-methyl]urea), a pharmaceutical formulation based on them and a method for their production and use on the basis of different therapeutic potency.

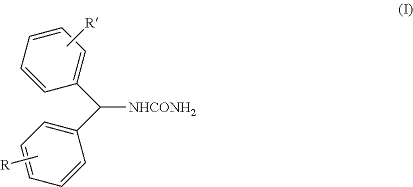

Where R≠R′ and are selected from the group hydrogen, alkyl, halogen, nitro, amino, alkyl amino and hydroxy groups and are found in ortho-, para-, or meta positions on benzene rings.

### BACKGROUND OF THE INVENTION

It is known that racemic benzhydrol ureas represented by formula (I) have anticonvulsant properties (RF patents 675790, 2070039, 2033412, 2033411 and 2024504). Among them is a product derived from racemic 1-[(3-chlorophenyl)-phenyl-methyl]urea, which is classified as an anticonvulsant and has received the international nonproprietary name “halodif.” All known methods for the production of benzhydrol ureas represented formula (I) result in racemic forms. For example, there is a method for halodif synthesis in which the end product is obtained by the condensation of 3-chlorophenyl-phenyl-methanol with urea by heating in the presence of concentrated sulfuric acid (SU 1833611 A3, Nov. 11, 1994). The disadvantage of the method is that the resulting halodif is a racemic mixture of optical isomers, although the therapeutic potency of the optical isomers may vary. There is a method for the production of m-chlorodiphenyl methyl urea (RF patent 2092478, IPC C07C275/24, May 28, 1992) based on the condensation of a derivative of m-chlorodiphenyl methane represented by the general formula:

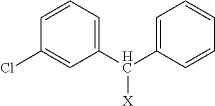

where X=—OH, —OCOCH₃, —OCOCF₃, —OS0₂Ph, —NH₂, —NH₂HCl, or —NHCOH, with urea in the presence of a mineral acid selected from the series H2SO4, HCl, HCIO₂, at 40-160° C. with the following reagent molar ratios: derivative of m-chlorodiphenyl methane urea mineral acid 1 (1.1-8.0) (0.01-1.5). The halodif produced by this method, however, is also a racemic mixture of optical isomers, the therapeutic potency of which may vary, which, when the racemic mixtures are used, may result in side effects, particularly in combination therapy.

These methods for the synthesis of benzhydrol ureas represented by formula (I), specifically 1-[(3-chlorophenyl)-phenyl-methyl]urea, do not produce individual optical isomers from the racemates or allow for the full development of the pharmacological properties of the optical isomers, which may consist in a different manifestations of their therapeutic effect.

Among the known methods for the resolution of racemates is a resolution method using diastereomers. The substance of the method may be expressed in the following diagram:

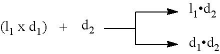

On the racemate to be resolved l₁·d₁ acts as the optically active reagent d₂: the result is a new pair of substances, l₁·d₂ and d₁·d₂, diastereomers differing in physical properties. The differences in solubility, vapor pressure and adsorption coefficients are in many cases are sufficient to separate the diastereomers by crystallization, distillation or chromatographically.

It is known that acidic asymmetric reagents are most commonly used to resolve racemic amines. These usually are (+)-tartaric acid (the cheapest and most readily available of the asymmetric reagents). (Teaching Materials, Organic Chemistry, Methods for the Production of Stereoisomers, Chemistry Faculty, Moscow State University, httts://www.chem.msu.su/rus/teaching/stereo/iii.html), RF patent 2132845 (IPC C07D211/90, Mar. 6, 1995) describes a method for isolating R −/+/− and S −/−/−isomers of amlodipine from mixtures thereof, which consists in the interaction of a mixture of the isomers with L- or D-tartaric acid in an organic solvent containing a quantity of dimethylsulfoxide /DMSO/ sufficient for the precipitation of a DMSO-solvate of a salt of L-tartaric acid and R−/+/−amlodipine or a DMSO-solvate of a salt of D-tartaric acid and S −/−/−amlodipine.

The general essential feature of this prior art is the isolation of the isomers by the interaction of a racemic mixture of the isomers with L- or D-tartaric acid in an organic solvent.

There is a known method for the production of (+) and (−) enantiomers of (3-chlorophenyl)-phenyl-methane amine by reacting racemic (3-chlorophenyl)-methane amine with (+) and (−) tartaric acids, followed by the neutralization of the resulting tartrate diastereomers with an aqueous ammonia solution in chloroform (U.S. Pat. No. 6,172,228 (B2)) and boiling off the solvent. The general essential feature of this prior art is the interaction of the racemic mixture of isomers (3-chlorophenyl)-phenyl-methane amine with L- or D-tartaric acid.

### SUMMARY OF THE INVENTION

The main objective of the disclosed group of inventions is to produce optical isomers of (+)- and (−) benzhydrol ureas represented by formula (I) and of (+)- and (−) [(3-chlorophenyl)-phenyl-methyl]urea and a pharmaceutical formulation derived therefrom and to develop a method for their production in order to determine the individual therapeutic potency of the optical isomers for further use. The technical result to be achieved by the application of the declared group of inventions is to expand the functional capabilities of anticonvulsants based on benzhydrol ureas represented by formula (I) and based on [(3-chlorophenyl)-phenyl-methyl]urea, which have the international nonproprietary name “halodif,” by producing optical isomers with varying therapeutic potency. The disclosed method for obtaining these optical isomers has higher manufacturing efficiency that the prior art under U.S. Pat. No. 6,172,228 (B2).

The objective is achieved by the method for the production of optical isomers of (+)- and (−) benzhydrol ureas represented by formula (1+) and (I−) as presented below.

The present invention proposes optical isomers of (+)-benzhydrol ureas represented by formula (1+) with positive rotation of the plane of polarization.

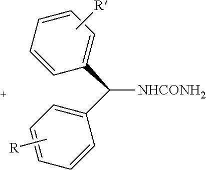

(1+) where R≠R′ and are selected from the group hydrogen, alkyl, halogen, nitro, amino, alkyl amino, hydroxy groups and are found in ortho-, para-, or meta-positions on benzene rings.

The present invention proposes optical isomers of (−)-benzhydrol ureas represented by formula (I−) with negative rotation of the plane of polarization.

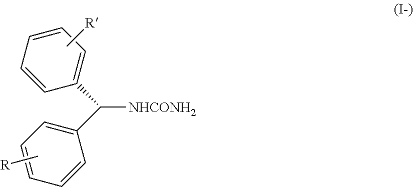

The present invention proposes optical isomers of (+)-1-[(3-chlorophenyl)-phenyl-methyl]urea with positive rotation of the plane of polarization.

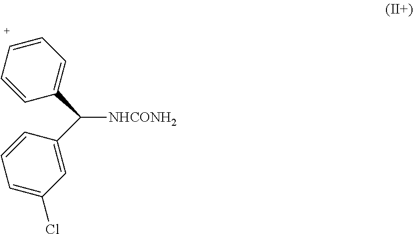

The present invention proposes optical isomers of (−)-1-[(3-chlorophenyl)-phenyl-methyl]urea with negative rotation of the plane of polarization. NHCONH2CI (II−).

The present invention proposes a pharmaceutical formulation having anticonvulsant action containing a therapeutically effective amount of at least one of the compounds corresponding to formulas (I+), (I−), (II+), (II−) in a mixture with at least one suitable carrier.

The term “pharmaceutically acceptable” denotes a group or a compound that is used to produce a pharmaceutical formulation and that is biologically or otherwise safe, nontoxic, and acceptable in both veterinary medicine and pharmaceutics.

The present invention proposes a method for the production of optical isomers of (+)- and (−)-benzhydrol ureas represented by formulas (1+) and (I−), in which the racemic mixture and benzhydrol amines represented by formula (III)

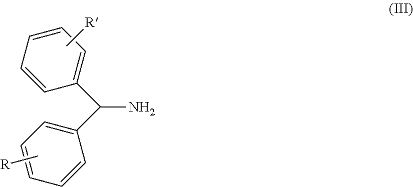

where R≠R′, selected from the group hydrogen, alkyl, halogen, nitro, amino, alkyl amino, hydroxy groups and are found in ortho-, para-, or meta-positions on benzene rings, are used to produce diastereomers of tartrates of benzhydrol amines represented by formula (III+)

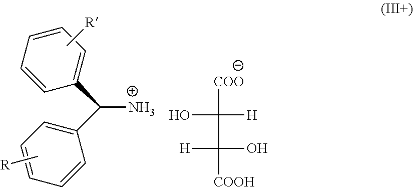

and represented by formula (III−)

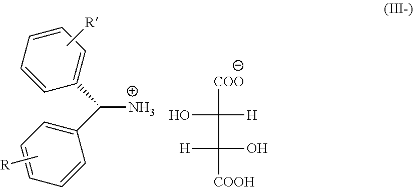

in the presence of tartaric acids in an organic solvent, while optical isomers of (+)- and (−)-benzhydrol ureas represented by formulas (1+) and (I−) are produced through the interaction of diastereomers of tartrates of benzhydrol amines represented by formula (III) and represented by formula (III−) with cyanates of alkaline metals in an aqueous solution.

The present invention proposes a method for the production of optical isomers of (+)- and (−)-benzhydrol ureas represented by formula (1+) and (I−), in which the interaction of diastereomers of tartrates (+)- and (−)-benzhydrol amines with an aqueous solution of cyanates of alkaline metals takes place at room temperature.

The present invention proposes a method for the production of optical isomers of (+)- and (−)-benzhydrol ureas represented by formula (1+) and (I−) in which the tartaric acids are L- or D-tartaric acid.

The present invention proposes a method for the production of optical isomers of (+)- and (−)-benzhydrol ureas represented by formula (1+) and (I−) in which the organic solvent is methanol or other aliphatic alcohols.

The present invention proposes a method for the production of optical isomers of (+)- and (−)-benzhydrol ureas represented by formula (1+) and (I−) in which the racemic mixture of benzhydrol amines represented by formula (III) is (3-chlorophenyl)-phenyl-methane amine.

The present invention proposes a method for the production of optical isomers of (+)- and (−)-benzhydrol ureas B in which a racemic mixture of (3-chlorophenyl)-phenyl-methane amine is used to produce (+)- and (−) diastereomers of tartrates of (3-chlorophenyl)-phenyl-methane amine in the presence of tartaric acids in an organic solvent.

The present invention proposes a method for the production of optical isomers of (+)- and (−)-[(3-chlorophenyl)-phenyl-methyl]urea in which a racemic mixture of (3-chlorophenyl)-phenyl-methane amine is used to produce (+)- and (−) diastereomers of tartrates of (3-chlorophenyl)-phenyl-methane amine in the presence of tartaric acids in an organic solvent, and (+)- and (−) diastereomers of tartrates (3-chlorophenyl)-phenyl-methane amine interact with cyanates of alkaline metals in an aqueous solution.

Unlike the prior art (U.S. Pat. No. 6,172,228), in the disclosed method there is no need to obtain (+) and (−) enantiomers of benzhydrol amines, inasmuch as, as FIG. 1 shows, the formation of (+) and (−) enantiomers [sic] of benzhydrol urea (I) is achieved by reacting cyanates of alkaline metals with diastereomers of the corresponding (+) and (−) tartrates of benzhydrol amines, which is a significant technological advantage and a novelty.

### BRIEF DESCRIPTION OF THE DRAWINGS

FIG. 1 presents a diagram of the method for the production of optical isomers (+)- and (−)-benzhydrol ureas represented by formula (1+) and (I−), based on the production, from a racemic mixture of the corresponding benzhydrol amines represented by formula (III), of diastereomers of the tartrates of those benzhydrol amines represented by formula (III+) and represented by formula (III−) in the presence of tartaric acids in an organic solvent and the interaction of the resulting (+)- and (−) diastereomers of tartrates benzhydrol amines with cyanates of alkaline metals in an aqueous solution.

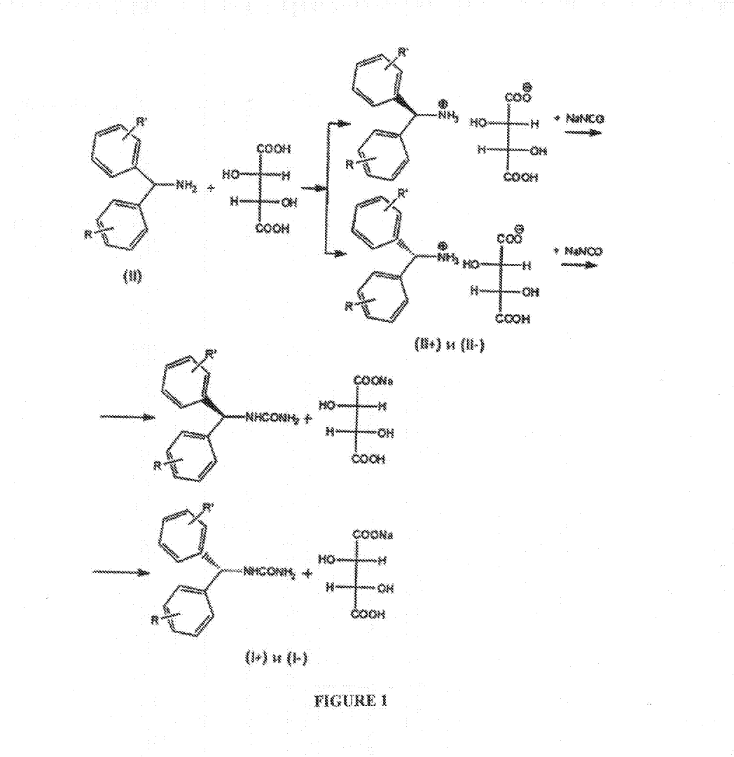

The invention is further illustrated by examples of its implementation which, however, do not limit possible alternatives for its implementation within the limits of the character and scope of the present invention.

### DETAILED DESCRIPTION OF THE PREFERRED EMBODIMENT

### Example 1

Production of diastereomeric salts of (3-chlorophenyl)-phenyl-methane amine. 4.5 g (0.03 mol) of D-(−)-tartaric acid were dissolved in 63 mL of methanol at a temperature of 60-65° C.; 6.4 g (0.03 mol) of racemic (3-chlorophenyl)-phenyl-methane amine were added to the hot methanol solution of D-(−)-tartaric acid, and heated at that temperature for another 5 minutes. Then the mixture of diastereomers was stirred for 3 hours without heating. The precipitated tartrate of (−)(−)-(3-chlorophenyl)-phenyl-methane amine was filtered and rinsed on the filter with 10 mL of methanol cooled to 10° C., and dried, after which it was recrystallized from the methanol. The yield of tartrate of (−)(−)-(3-chlorophenyl)-phenyl-methane amine was 5.3 g (48%) expressed in terms of a theoretical mass of racemic amine. Melting point: Tₘ=160-162° C. ¹H Nuclear magnetic resonance spectroscopy (NMRS):

¹H NMRS (300 MHz, D₂0), δ=5.6s (3H, NH₃⁺), 7.3 (1H, CH), 7.4 (9H, Ag), 4.4 (2H, CH).

¹³C Nuclear magnetic resonance spectroscopy:

¹³C NMRS (300 MHz, DMSO-d₆), δ=: 130, 128.5, 126.5, 125, 72, 57.

The filtrate containing tartrate of (−)(+)-(3-chlorophenyl)-phenyl-methane amine was boiled down after the methanol was boiled off. Then it was cooled to room temperature, and the precipitate of the tartrate of (−)(+)-(3-chlorophenyl)-phenyl-methane amine was filtered and rinsed on the filter with 5 mL of toluene and dried, after which it was recrystallized from the methanol. The yield of tartrate of (−)(+)-(3-chlorophenyl)-phenyl-methane amine was 5.3 g (48%) expressed in terms of a theoretical mass of racemic amine.

Tₘ=143-145° C.

The NMR spectra of the tartrates (−)(−)- and (−) (+)-(3-chlorophenyl)-phenyl-methane amine coincide.

Production of (+)- and (−)-1-[(3-chlorophenyl)-phenyl-methyl]urea. A solution of 2.1 g (0.032 mol) of sodium cyanate in 24 mL of water was added to a solution of 5.3 g (0.014 mol) of tartrate of (−) (−)-(3-chlorophenyl)-phenyl-methane amine in 30 mL of ethanol. The mixture was stirred for 1 hour at room temperature. The end of the reaction was monitored by TLC method. After cooling, water was added to the reaction mass until the (−)-1-[(3-chlorophenyl)-phenyl-methyl]urea fully precipitated, after which it was filtered, rinsed with water, dried and cleaned by dissolving in ethyl acetate and precipitating with hexane. The yield of (−)-1-[(3-chlorophenyl)-phenyl-methyl]urea was 3.1 g (85%). Acicular crystals formed during the crystallization of the (−)-1-[(3-chlorophenyl)-phenyl-methyl]urea from the aqueous solution.

### Melting Point:

Tₘ-137-138° C.

### Specific Rotation:

[α]_{D}²⁰−5.000 (ethanol, c 6.6).

¹H Nuclear magnetic resonance spectroscopy: ¹H NMR (300 MHz, DMSO-d₆), δ=5.6s (2H, NH₂), 5.9 (1H, CH), 7.0 (1H, NH), 7.3 (6H, Ag).

¹³C Nuclear magnetic resonance spectroscopy:

¹³C NMRS (300 MHz, DMSO-d₆), δ=158, 146, 143, 133, 130, 128, 127, 126, 125, 56.

### Infrared Spectroscopy:

IR (KBr) ν/CM⁻¹: 3440 (NH₂); 3340 (NH); 1650 (C=0).

### High-Efficiency Liquid Chromatography (HELC):

Agilent 1200 Compact LC, column: 150×4.6 mm, stationary phase: ZorbaxExtend C-18 (5 μm), eluent: acetonitrile-water (gradient eluation, acetonitrile-water ratio at the beginning of the test 0%:100%; at the end of the test 100%: 0%); eluent flow speed: 1.0 mL/min; detection at a wavelength of 230 nm; injection volume—20 μL (injection loop), hold time of (−)-halodif 7.23 min.

(+)-1-[(3-chlorophenyl)-phenyl-methyl]urea was produced from tartrate of (−)(+)-(3-chlorophenyl)-phenyl-methane amine in a manner similar to the method presented above. The yield of (+)-1-[(3-chlorophenyl)-phenyl-methyl]urea was 3.1 g (85%). Cubic crystals from during the crystallization of the (+)-1-[(3-chlorophenyl)-phenyl-methyl]urea from aqueous ethanol.

Tₘ=137-138° C. [α]_{D}²⁰+5.00^{e} (ethanol, c 6.6).

The NMR and IR spectra, and the HELC hold time for the (−)- and (+)-1-[(3-chlorophenyl)-phenyl-methyl]urea coincide.

### Example 2

### Production of diastereomeric salts of (4-chlorophenyl)-phenyl-methane amine

4.5 g (0.03 mol) of D-(−)-tartaric acid were dissolved in 63 mL of methanol at a temperature of 60-65° C. 6.4 g (0.03 mol) of racemic (4-chlorophenyl)-phenyl-methane amine were added to the hot methanol solution of D-(−)-tartaric acid. Heating at that temperature continued for another 5 minutes. Then the mixture of diastereomers was stirred for 3 hours without heating. The precipitated tartrate of (−)(−)-(4-chlorophenyl)-phenyl-methane amine was filtered and rinsed on the filter with 10 mL of methanol cooled to 10° C. and dried, after which it was recrystallized out of the methanol. The yield of tartrate of (−)(−)-(4-chlorophenyl)-phenyl-methane amine was 5.4 g (49%) expressed in terms of a theoretical mass of racemic amine.

### Melting Point:

Tₘ=179-180° C.

### ¹H Nuclear Magnetic Resonance Spectroscopy:

¹H NMR (300 MHz, D₂0), δ=5.6s (3H, NH₃+), 7.3 (1H, CH), 7.4 (9H, Ag), 4.4 (2H, CH).

### ¹³C Nuclear Magnetic Resonance Spectroscopy:

¹³C NMR (300 MHz, DMSO-d₆), δ=130, 128.5, 126.5, 125, 72, 57.

A filtrate containing a tartrate of (−)(+)-(4-chlorophenyl)-phenyl-methane amine was boiled down by boiling off the methanol, after which it was cooled to room temperature. The precipitate of the tartrate of (−)(+)-(4-chlorophenyl)-phenyl-methane amine was filtered, rinsed on the filter with 5 mL of toluene and dried, after which it was recrystallized from the methanol. The yield of tartrate of (−)(+)-(4-chlorophenyl)-phenyl-methane amine was 5.4 g (49%) expressed in terms of a theoretical mass of racemic amine.

Tₘ=163-164° C.

The NMR spectra of the tartrates of (−)(−)- and (−)(+)-(4-chlorophenyl)-phenyl-methane amine coincide.

### Production of (+)- and (−)-1-[(4-chlorophenyl)-phenyl-methyl]urea

A solution of 2.1 g (0.032 mol) of sodium cyanate in 24 mL of water was added to a solution of 5.3 g (0.014 mol) of tartrate of (−)(−)-(4-chlorophenyl)-phenyl-methane amine B 30 mL of ethanol. The mixture was stirred for 1 hour at room temperature. The end of the reaction was monitored by TLC method. After cooling, water was added to the reaction mass until the full precipitation of the (−)-1-[(4-chlorophenyl)-phenyl-methyl]urea, which was filtered, rinsed with water, dried and cleaned by dissolving it in ethyl acetate and precipitating with hexane. The yield of (−)-1-[(4-chlorophenyl)-phenyl-methyl]urea was 3.2 g (88%). Acicular crystals formed during the crystallization of the (−)-1-[(4-chlorophenyl)-phenyl-methyl]urea from aqueous ethanol.

### Melting Point:

Tₘ=153-154° C.

### Specific Rotation:

[α]_{D}²⁰−8.73° (ethanol, c 6.6).

### ¹H Nuclear Magnetic Resonance Spectroscopy:

¹H NMS (300 MHz, DMSO-d₆), δ=5.6s (2H, NH₂), 5.9 (1H, CH), 7.0 (1H, NH), 7.3 (6H, Ag).

### ¹³C Nuclear Magnetic Resonance Spectroscopy:

¹³C NMR (300 MHz, DMSO-d₆), δ=158, 146, 143, 133, 130, 128, 127, 126, 125, 56.

### IR Spectroscopy:

IR (KBr) ν/CM⁻¹: 3440 (NH₂); 3340 (NH); 1650 (C=0).

### High-Efficiency Liquid Chromatography (HELC):

Agilent 1200 Compact LC, column: 150×4.6 mm, stationary phase: ZorbaxExtend C-18 (5 μm), eluent acetonitrile-water (gradient eluation, acetonitrile-water ratio at the beginning of the test 0%:100%; at the end of the test 100%: 0%); eluent flow speed: 1.0 mL/min; detection at a wavelength of 230 nm; injection volume—20 μL (injection loop), hold time of (−)-halodif 6.4 min.

(+)-1-[(3-chlorophenyl)-phenyl-methyl]urea was produced from tartrate of (−)(+)-(4-chlorophenyl)-phenyl-methane amine in a manner similar to the method presented above. The yield of (+)-1-[(4-chlorophenyl)-phenyl-methyl]urea was 3.1 g (85%). Cubic crystals formed during the crystallization of the (+)-1-[(4-chlorophenyl)-phenyl-methyl]urea from aqueous ethanol.

Tₘ=153-154° C. [α]_{D}²⁰−8.73° (ethanol, c 6.6).

The NMR and IR spectra, and the HELC hold time for the (−)- and (+)-1-[(4-chlorophenyl)-phenyl-methyl]urea coincide.

The therapeutic potency of the resulting optical isomers of (+) and (−)-1-[(3-chlorophenyl)-phenyl-methyl]urea (halodif) was studied.

Convulsions were induced by the administration of pentylenetetrazol. The severity (stage) of the convulsions was rated on a five-point scale. The anticonvulsant potency was rated on the basis of the substance's ability to reduce the severity of convulsions induced by the pentylenetetrazol. Halodif substances were administered intragastrically over 2 hr prior to the pentylenetetrazol. The pentylenetetrazol was administered subcutaneously. Probit analysis was used for statistical processing of the results with Student's test, ANOVA and criterion x. The differences in the results were considered statistically reliable at a significance level of p<0.05.

In the first phase of the study the optimum convulsion-inducing dose of pentylenetetrazol, CD97, which was 110 mg/kg (Table 1) was established.

TABLE 1
Number of animals with stage 3 convulsions as a function of the
pentylenetetrazol dose.

| | | | Proportion of |
|---|---|---|---|
| | Number of | Number of animals | animals |
| Pentylenetetrazol | animals in the | with stage 3 | with stage |
| dose | group | convulsions | 3 (%) |
| 80 | 5 | 0 | 0 |
| 90 | 5 | 3 | 60 |
| 100 | 5 | 4 | 80 |
| 110 | 5 | 4 | 80 |

In the second phase the anticonvulsant potency of the reference substance was study in doses of 50 mg/kg, 100 mg/kg, 150 mg/kg, 200 mg/kg and 500 mg/kg (Table 2). The reference substance was racemic halodif. Three doses were selected to conduct a comparative study of halodif specimens on the basis of the results: 100 μg/kg, 150 μg/kg and 250 μg/kg. The halodif reference substance was used in doses of 50 mg/kg, 100 mg/kg, 150 mg/kg, 200 mg/kg and 500 mg/kg. Two hours after the halodif substance was administered to the animals the pentylenetetrazol (110 mg/kg) was administered and stage 3 and stage 5 convulsions were rated. Table 2 presents the results.

TABLE 2
Effect of different doses of the halodif reference substance on the number
of animals with stage 3 and stage 5 convulsions.

| | | Proportion |
|---|---|---|
| | Proportion of animals with | of animals with |
| Dose of reference | stage 3 convulsions (%, | stage 5 convulsions (%, |
| substance (mg/kg) | n = 5) | n = 5) |
| 50 | 100 | 80 |
| 100 | 100 | 20 |
| 150 | 70 | 10 |
| 200 | 60 | 0 |
| 500 | 60 | 0 |

The studies showed that the halodif reference substance prevented the development of clonic convulsions (stage 3 convulsions) in 30-40% of the animals starting at a dose of 150 μg/kg (table 2). The reference substance protected 20% of the animals from tonic convulsions in all extremities (stage 5) in the lowest dose (50 mg/kg). When the dose was increased to 200 mg/kg and higher, the reference substance completely prevented the appearance of convulsions in this stage.

Three doses of the product were selected on the basis of the results to perform a comparative study of samples of (+) and (−)-halodif:
the maximum dose that does not affect clonic convulsions—100 μg/kg;
the minimum dose that reduces the number of animals with clonic convulsions—150 μg/kg;
a dose of 250 μg/kg to ascertain the sample with the highest potency.

To compare anticonvulsant potency the specimens of halodif substances were administered in three doses: 100 mg/kg, 150 mg/kg and 250 mg/kg 2 hours before pentylenetetrazol (110 mg/kg).

Samples were compared on the basis of the mean score for convulsive manifestations induced by pentylenetetrazol after administration of the samples under study. Table 3 presents the results.

TABLE 3
Influence of the reference substance and samples of substances of
(+) and (−) - isomers of halodif on convulsive manifestations
(points) induced by pentylenetetrazol (X ± SE)

| | Dose of sample study (mg/kg) | | |
|---|---|---|---|
| Sample of halodif substance | the under | | |
| under study | 100 | 150 | 250 |
| Sample 1/(−) isomer | 3.8 ± 0.39 | 2.9 ± 0.29 | 2.8 ± 0.41 |
| Sample 2/(−) isomer | 4.1 ± 0.23* | 3.5 ± 0.22* | 3.0 ± 0.37 |
| Sample 3/(+) isomer | 3.2 ± 0.42 | 2.5 ± 0.31 | 1.9 ± 0.28 |
| Sample 4/(+) isomer | 3.0 ± 0.21 | 2.6 ± 0.31 | 2.5 ± 0.27 |
| Reference sample | 3.5 ± 0.31 | 2.9 ± 0.28 | 2.6 ± 0.33 |

Note:
*the difference is reliable in comparison with the corresponding figures for the control group (n = 10, p < 0.05).

Table 3 shows that, the higher the dose of the product, the lower the mean score for convulsions. For a dose of sample 1 equal to 100 (mg/kg), the mean convulsions score was 3.8±0.39, while for the same sample 1 in a dose of 250 (mg/kg), the mean convulsions score fell to 2.8±0.41.

A comparison of data from table 3 for the reference sample and samples 1 and 2 of the ((−)-isomer of halodif) shows that the mean convulsions score for samples 1 and 2 for all doses of the product above was higher than that of the reference sample, i.e., the (−)-isomer of halodif exhibited less anticonvulsive potency than did the reference substance. On the basis of the mean convulsions scores, samples 3 and 4 in all doses studied: 100 mg/kg, 150 mg/kg, 250 mg/kg had a lower convulsions score, i.e., the (+)-isomer of halodif exhibited more anticonvulsive potency than did the reference substance (p<0.05).

The anticonvulsive potency of the samples of the halodif substance under study were also compared with respect to the proportion of animals in which the substance under study prevented the development of convulsions of differing severity induced by pentylenetetrazol. Table 4 presents the results expressed as the percentage of animals in which no convulsions of the specified stage developed (% protection).

TABLE 4
Comparison of the samples under study in different doses with respect to
their ability to protect animals (% protection) against stage
3, 4 and 5 convulsions induced by pentylenetetrazol

| | Dose of the | | |
|---|---|---|---|
| | sample (mg/kg) | | |
| Substance sample of halodif | 100 | 150 | 250 |

Protection against stage 3

| | | | |
|---|---|---|---|
| Sample 1/(−) isomer | 10 | 20 | 40 |
| Sample 2/(−) isomer | 10 | 10 | 30 |
| Sample 3/(+) isomer | 30 | 40 | 70 |
| Sample 4/(+)isomer | 20 | 30 | 50 |
| Reference sample | 20 | 30 | 50 |

Protection against stage 4

| | | | |
|---|---|---|---|
| Sample 1/(−) isomer | 40 | 70 | 80 |
| Sample 2/(−) isomer | 20* | 40* | 70 |
| Sample 3/(+) isomer | 60 | 90 | 100 |
| Sample 4/(+) isomer | 80 | 90 | 90 |
| Reference sample | 50 | 70 | 80 |

Protection against stage 5

| | | | |
|---|---|---|---|
| Sample 1/(−) isomer | 60* | 80 | 100 |
| Sample 2/(−) isomer | 70 | 70 | 100 |
| Sample 3/(+) isomer | 80 | 100 | 100 |
| Sample 4/(+) isomer | 100 | 100 | 100 |
| Reference sample | 80 | 90 | 100 |

Note:
*the difference is reliable in comparison with the corresponding figures for the control group (n = 10, p < 0.05).

Table 4 shows that the proportion of animals protected against stage 3, 4 and 5 convulsions for all samples under study increases with an increase in the dose of halodif. Samples 3 and 4, which correspond to the (+)-isomer of halodif, demonstrated the greater protection against convulsions at all stages. Racemic halodif offered reliably lower protection than did samples 3 and 4, the ((+)-isomer of halodif), and reliably higher protection than samples 1 and 2, ((−)-isomer of halodif), in doses of 100 mg/kg and 150 mg/kg for stages 3 and 4.

All the samples fully protected the animals against the development of stage 5 convulsions in a dose of 250 mg/kg.

The results of the experiments confirm that optical isomers of 1-[(3-chlorophenyl)-phenyl-methyl]urea (isomers of halodif) produced by the method according to the invention may be used as anticonvulsive medicinal products.

### INDUSTRIAL APPLICABILITY

These tests therefore established that the optical isomer (+)-1-[(3-chlorophenyl)-phenyl-methyl]urea (+) isomer of halodif) has higher anticonvulsive potency than its optical antipode, (−)-1-[(3-chlorophenyl)-phenyl-methyl]urea, the ((−) isomer of halodif). Separating racemic halodif 1-[(3-chlorophenyl)-phenyl-methyl]urea by the method according to the invention produced optical isomers of (+)- and (−)-1-[(3-chlorophenyl)-phenyl-methyl]urea (isomers of halodif) with differing therapeutic potencies.

1. An optical isomers of (+)-1-[(3-chlorophenyl)-phenyl-methyl]urea represented by formula (1+) with positive rotation of the plane of polarization.

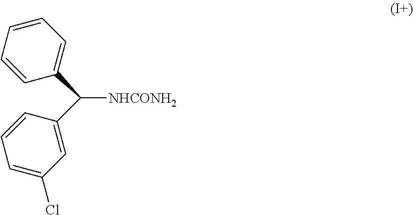

2. An optical isomers of (−)-1-[(3-chlorophenyl)-phenyl-methyl]urea represented by formula (I−) with negative rotation of the plane of polarization.

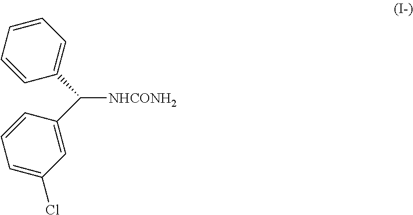

3. A pharmaceutical formulation with anticonvulsive action, containing a therapeutically potent quantity of at least one of the compounds corresponding to formulas (I+) and (I−) in claim 1 and claim 2 in a mixture with at least one suitable carrier.

4. A method for the production of optical isomers of (+)- and (−)-1-[(3-chlorophenyl)-phenyl-methyl]urea in which a racemic mixture of benzhydrol amines represented by formula (II) 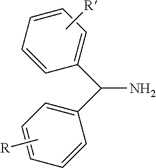
where R≠R′, and selected from the group hydrogen and halogen found in the ortho-, para-, or meta-positions of benzene rings, are used to produce diastereomers of tartrates of benzhydrol amines represented by formula (II+) 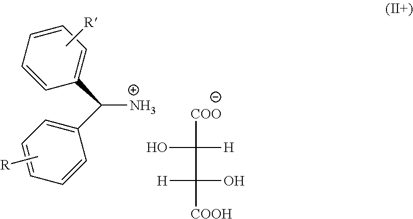
and formula (II−) 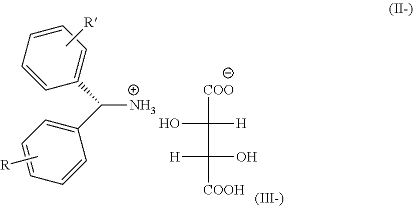
in the presence of tartaric acids in an organic solvent, and optical isomers of (+)- and (−)-1-[(3-chlorophenyl)-phenyl-methyl]urea represented by the formulas (I+) and (I−) are obtained by the interaction of diastereomers of tartrates of benzhydrol amines represented by the formula (II+) and formula (II−) with cyanates of alkaline metals in an aqueous solution.

5. The method as set in claim 4, wherein the interaction of diastereomers of tartrates of (+)- and (−)-benzhydrol amines with aqueous solutions of cyanates of alkaline metals takes place at room temperature.

6. The method as set in claim 4, wherein L- or D-tartaric acid is selected as the tartaric acid.

7. The method as set in claim 4, wherein methanol or other aliphatic alcohols are selected as the organic solvent.

8. The method as set in claim 4, wherein the (3-chlorophenyl)-phenyl-methane amine is selected as the racemic mixture of benzhydrol amines represented by formula (III).

9. The method as set in claim 8, wherein that (+)- and (−) diastereomers of tartrates of (3-chlorophenyl)-phenyl-methane amine are produced from the racemic mixture of (3-chlorophenyl)-phenyl-methane amine in the presence of tartaric acids in an organic solvent.

10. The method as set in claim 9, wherein the (+)- and (−) diastereomers of tartrates of (3-chlorophenyl)-phenyl-methane amine interact with cyanates of alkaline metals in an aqueous solution to product optical isomers of (+)- and (−)-1-[(3-chlorophenyl)-phenyl-methyl]urea.
